(19) United States
(12) Patent Application Publication
(10) Pub. No.: US 2021/0268285 A1
(43) Pub. Date: Sep. 2, 2021

(54) ELECTROMECHANICAL IMAGING

(71) Applicant: KONINKLIJKE PHILIPS N.V., EINDHOVEN (NL)

(72) Inventor: Shlomo BEN-HAIM, EINDHOVEN (NL)

(21) Appl. No.: 17/257,402

(22) PCT Filed: Jul. 4, 2019
(86) PCT No.: PCT/EP2019/068052

§ 371 Date: Dec. 31, 2020

Related U.S. Application Data
(60) Provisional application No. 62/693,943, filed on Jul. 4, 2018.

(51) Int. Cl. A61N 1/368; A61N 1/372; A61N 1/05

(52) U.S. Cl. CPC A61N 1/3686; A61N 1/372; A61N 2001/0585; A61N 1/0587; A61N 1/056

(57) ABSTRACT

A method for selecting an implantation position of a pacing electrode of a pacing lead within an anatomical region of a body, comprising measuring values of at least one parameter of an anatomical region surrounding the pacing lead in at least one location. The method also comprises delivering an electric field to a tissue of the anatomical region, and measuring values of at least one parameter of the tissue surrounding the pacing lead in the at least one location after said delivering. The method further comprises determining changes in said measured values before and after said delivering and selecting an implantation position for the pacing electrode based on said determined changes.

### FIELD OF THE INVENTION

The invention relates to methods, apparatus, systems and computer program product for. The present invention, in some embodiments thereof, relates to sensing and, more particularly, but not exclusively, to sensing in blood vessels and/or in the heart and/or on the heart such as to guide the implementation of pacing electrode.

### BACKGROUND OF THE INVENTION

In many interventions to treat heart disease the knowledge of some of the tissue specific local anatomy and or electrical activation and or related mechanical contraction and or the electro-mechanical coupling can improve procedure efficiency and efficacy.

Specifically, for the treatment of heart failure, physician commonly implant a “resynchronization” pacemaker to improve the function of the failing heart. In this treatment a physician usually implant two pacing leads that activate the left ventricle in an “optimal” way. One of these leads is usually placed in over the left ventricle epicardium. The physician is accessing the left ventricle epicardium through cannulation of the pacing lead via the coronary sinus. The left ventricular pacing lead is positioned in an optimal epicardial vein that originate from the coronary sinus. During the implantation the physician has to follow a sequence of steps to allow him identify the optimal location for the left ventricular lead placement. These steps include injecting contrast media under fluoroscopy into the coronary sinus when an inflated balloon assuring the spread into the coronary vein in a retrograde manner. Inflating a balloon in the coronary sinus vein can lead to its rupture, a life threatening medical emergency. The road map of the coronary venous tree is captured using this venography. Following that step the physician threads the pacing lead to one of the coronary vein branches to test its suitability for implanting the lead. Among the criteria the physician need to assess is its anatomical location, presence of a viable left ventricular myocardium underneath the epicardial vein, the timing of its local epicardial electrical activation, and its local electromechanical coupling. Currently the tools the physician can use to guide threading the lead require using X-Ray radiation, in some cases of CRT lead implantation physician use more than 30 minutes of fluoroscopy exposure. Furthermore, the physician has to inject a contrast agent to the patient blood which for some patients (especially those with failing heart) can increase the likelihood for kidney failure. The physician can record the local activation at different sites but he does not have the tool to assist him in mapping epicardial electrical activation. In addition, the physician does not have tool to map left ventricular myocardial viability as well as the electromechanical coupling of the left ventricle.

The efficacy of CRT implantation for the treatment of heart failure is below 50%. Nevertheless, as these patients have no other option physician still try and use this therapy knowing of its low efficacy, and low efficiency.

### SUMMARY OF THE INVENTION

It is an object of the invention to provide a safe, let alone accurate methods, apparatus, systems and computer program products to guide the implementation position of one or more pacing electrodes coupled to a pacing lead in a region of interest, such region preferably being part of the cardiovascular system, for instance the left ventricle.

According to an aspect of the invention, this object is realized by a method for selecting an implantation position of a pacing electrode of a pacing lead within an anatomical region of a body, comprising: measuring values of at least one parameter of an anatomical region surrounding the pacing lead in at least one location; delivering an electric field to a tissue of the anatomical region; measuring values of at least one parameter of the tissue surrounding the pacing lead in the at least one location after said delivering; determining changes in said measured values before and after said delivering; and selecting an implantation position for the pacing electrode based on said determined changes.

According to a further aspect of the invention, the object is realized by an apparatus for selecting an implantation position of a pacing electrode of a pacing lead within an anatomical region, the apparatus comprising a processor circuit in communication with the pacing lead, wherein the processor circuit is configured to (i) measure values of at least one parameter of a tissue surrounding the pacing lead in at least one location within the anatomical region; (ii) deliver an electric field to a cardiac tissue; (iii) measure values of at least one parameter of the tissue surrounding the pacing lead in the at least one location within the anatomical region after said delivering; (iv) determine changes in said measured values before and after said delivering and (v) select an implantation position for the pacing electrode of the pacing lead based on said determined changes.

According to a further aspect of the invention, the object is realized by a system for selecting an implantation position of a pacing electrode within an anatomical region of a body, wherein the system comprises (i) the apparatus according to an aspect of the present invention; and the pacing lead coupled to at least a pacing electrode.

According to a further aspect of the invention, the object is realized by an computer program product comprising a computer readable medium having computer readable code embodied therein, the computer readable code being configured such that, on execution by a suitable computer or processor, the computer or processor is caused to perform the method according to an aspect of present invention

It is an advantage of the invention to provide for lead placement at a determined location within the region of interest which is safer for the users, such as the patient, the physician, the hospital personal. The present invention allow the implantation of one or more pacing electrode with lower dose of fluoroscopy or completely withdraw the need for fluoroscopy. The X-Ray radiations are therefore significantly diminished, where the insertion of contrast agent (or dye) into the patient also reduced, or totally removed. Accordingly, the implantation procedure may be advantageously guided without the use of fluoroscopy, angiography, and/or contrast agent.

The invention is further advantageous is that it enable the determination of an optimal left ventricular (LV) pacing site in real-time, regardless as if the subject is classified as a responder or non-responder.

The invention is further advantageous as it allow the guiding of the pacing lead from within the coronary vein tree without the need for other intravascular device, nor other pre-acquired images; thereby decreasing the procedure time, decreasing the procedure time and improving.

According to an embodiment, the anatomical region comprises a region of the cardiovascular system, preferably the epicardium, endocardium, coronary vein and/or left ventricle of the heart.

According to an embodiment, the at least one parameter comprises impedance, conductivity and/or thickness.

According to an embodiment, the method further comprises generating an anatomical and/or functional map of the anatomical region and selecting the implantation position of the pacing electrode based on said anatomical and/or functional map. This embodiment is advantageous as it allows an increase of accuracy of the navigating and positioning of the pacing lead within the anatomical region and/or within the cardiovascular system, thereby safeguarding patient safety and speed of the procedure.

According to an embodiment, the generating of the anatomical and/or functional map comprises: moving the pacing lead within a bodily cavity of the anatomical region; measuring values of at least one parameter of a tissue surrounding said pacing lead at one or more locations within said bodily cavity over time; calculating changes in said measured values before and after said delivering; mapping said anatomical region based on said calculated change of said measure value.

According to an embodiment, the method further comprising determining a geometrical change of the anatomical region and selecting the implantation position of the pacing electrode based of said geometrical change.

According to an embodiment, the determining of the geometrical change comprises, measuring values of the at least one parameter of a tissue surrounding the pacing electrode in at least two different locations within the anatomical region; determining a relative position of said two different locations based on said measured values; calculating a change in said determined position over a period of time in said two different locations; and determining a geometrical change of the anatomical region between said two different locations based on said calculated change in said determined position

According to an embodiment, the method further comprising synchronizing said measured parameter values with cardiac activity of the body and selecting the implantation position of the pacing electrode based on said synchronized parameter.

According to an embodiment, said determining comprises determining a position of said pacing electrode in relation to said cardiac activity.

According to an embodiment, the measuring is done by one or more electrodes coupled to the pacing lead, for instance a wireless pacing electrode (lead-less).
According to an embodiment, the processing circuit is configured to generate an anatomical and/or functional map of the anatomical region and selecting the implantation position of the pacing electrode based on said anatomical and/or functional map.

According to an embodiment, the processing circuit is configured to determine a geometrical change of the anatomical region and selecting the implantation position of the pacing electrode based of said geometrical change.

According to a further aspect of the invention, the object is realized by a system for selecting an implantation position of a pacing electrode within an anatomical region of a body, wherein the system comprises (i) the apparatus according to an aspect of the present invention; and the pacing lead coupled to at least a pacing electrode.

According to a further aspect of the invention, the object is realized by an computer program product comprising a computer readable medium having computer readable code embodied therein, the computer readable code being configured such that, on execution by a suitable computer or processor, the computer or processor is caused to perform the method according to an aspect of present invention

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as determining a position of a pacing lead, might be expected to use completely different methods, e.g., making use of expert knowledge and/or the pattern recognition capabilities of the human brain, which would be vastly more efficient than manually going through the steps of the methods described herein.

These and other aspects of the invention are apparent from and will be elucidated with reference to the embodiments described hereinafter.

It will be appreciated by those skilled in the art that two or more of the above-mentioned options, implementations, and/or aspects of the invention may be combined in any way deemed useful

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of a system for navigation and mapping, according to some embodiments of the invention;

FIG. 2 is a flow chart of a process for navigation and mapping, according to some embodiments of the invention;

FIG. 3 is a schematic illustration of blood vessels bifurcation identification, according to some embodiments of the invention;

FIG. 4 is a flow chart of a process for determining location in relation to cardiac cycle phase, according to some embodiments of the invention;

FIG. 5 is a schematic illustration of epicardial regions mapping, according to some embodiments of the invention;

FIG. 6 is a schematic illustration of electrical parameter recording following electric field delivery, according to some embodiments of the invention; and

FIG. 7 is a schematic illustration of an epicardial activation map, according to some embodiments of the invention.

### DESCRIPTION OF SPECIFIC EMBODIMENTS OF THE INVENTION

Certain embodiments will now be described in greater details with reference to the accompanying drawings. In the following description, like drawing reference numerals are used for like elements, even in different drawings. The matters defined in the description, such as detailed construction and elements, are provided to assist in a comprehensive understanding of the exemplary embodiments. Also, well-known functions or constructions are not described in detail since they would obscure the embodiments with unnecessary detail. Moreover, expressions such as “at least one of”, when preceding a list of elements, modify the entire list of elements and do not modify the individual elements of the list.

The present invention, in some embodiments thereof, relates to sensing and, more particularly, but not exclusively, to sensing in blood vessels and/or in the heart and/ or on the heart. In some embodiments the present invention relates to sensing using pacemaker lead, for example to aid in stimulator placement, for example for left ventricle (LV) lead placement.

An aspect of some embodiments relates to navigating within a body lumen using a pacemaker electrode lead. In some embodiments of the invention, the lead pacing electrodes and/or electrodes on a sheath thereof are used to identify bifurcations, locations and/or image, to aid such navigation. In some embodiments, the pacemaker electrode lead is navigated through elongated blood vessels, for example veins and/or arteries, for example coronary veins and/or arteries. Alternatively or additionally, the pacemaker electrode lead is navigated through body lumens of the heart. Optionally, the pacemaker electrode lead is navigated to an electrophysiological desired region suitable for implantation of an electric field transmitting electrode, for example a pacing electrode configured to be used in heart pacing procedures or a defibrillation electrode. In some embodiments of the invention, the pacing comprises one or more of endocardial pacing His Bundle pacing and CRT (cardiac resynchronization treatment).

According to some embodiments, the position of the pacemaker lead is determined during the navigation. In some embodiments, the position of the lead is determined based on measurements of at least one electrical parameter, for example conductivity and/or impedance. Optionally, the impedance and/or conductivity are of muscle tissue and/or of blood filled volumes and/or other tissue. In some embodiments, the position of the lead is determined using dielectric imaging. Exemplary dielectric imaging methods are described in PCT Patent Application IB 2018/050192 of Dichterman et al titled “Systems And Methods For Reconstruction Of Intra-Body Electrical Readings To Anatomical Structure”. In some embodiments, the position of the lead is determined using transmitting and/or receiving electrodes provided on the pacemaker lead. A potential advantage of the invention, is that it allows to perform navigation without contrast material and/or with at most 10, preferably 2 x-ray acquisitions, for example less than 30, 20, 10, 5, 1, 0.1,0.01 seconds of irradiation.

In some embodiments of the invention, a series of images are acquired and combined to form a model and a position may be determined by determining a position of a catheter relative to the model.

According to some embodiments, blood vessels bifurcations are identified during the pacemaker electrode lead navigation. In some embodiments, the bifurcations are identified by determining the position of the electrode lead during the navigation and/or using imaging analysis information. In some embodiments, the bifurcations are identified based on changes in the electrical properties of a tissue near the electrode lead, for example changes in dielectric, conductance, impedance properties of the tissue. In some embodiments, the bifurcations are identified by identifying an opening or a void in a generated image located at the side of the blood vessel.

In some embodiments of the invention, bifurcations are compared to a previously provided anatomical map, for example, based on a CT, MRI, ultrasound or x-ray image. It is noted that it is a particular feature of some embodiments of the invention that no such image is required, for example, in embodiments where the lead is used both to detect a location within a tree (and possibly map the tree) and determine an effect of electrification at one or more points.

According to some embodiments, lead electrodes, for example pacemaker lead electrodes are used for identifying bifurcations and/or to determine the position of the pacemaker lead. Optionally, one or more non-pacing electrodes are used, for example, part of the lead and/or one or more electrodes positioned on a sheath of the pacemaker lead. In some embodiments, sensing by the pacemaker lead is used to reconstruct an image (e.g., 1D, 2D and/or 3D) of tissue near the electrode, for example tissue located up to 1-7 cm, for example up to 2 cm, up to 3 cm, up to 5 cm or any intermediate, smaller or larger value from the electrode. In some embodiments of the invention, the imaging includes sections of tissue that are at least 1 cm, 3 cm, 5 cm and/or smaller or intermediate distances away from the electrodes on the lead. Exemplary methods for reconstruction an image of tissue are described in the above mentioned PCT Patent Application IB 2018/050192, and in U.S. Provisional Application No. 62/546,775 filed Aug. 17, 2017, titled “Field-gradient based remote imaging”.

An aspect of some embodiments relates to mapping tissue properties, for example cardiac tissue, or other tissue of other organs, such as the stomach or liver using a catheter, optionally a pacemaker electrode lead during an implantation process. In some embodiments, the cardiac tissue comprises epicardial and/or endocardial cardiac tissue. In some embodiments, a pacing electrode lead is navigated through blood vessels, for example arteries and/or veins located near the cardiac tissue. Optionally, the arteries and/or the veins are in contact with the cardiac tissue. In some embodiments, the tissue properties comprise electrical and/or mechanical properties. In some embodiments, a heart function is determined based on said measured tissue properties.

According to some embodiments, one or more electrodes of the pacing lead measure values of at least one electrical parameter, for example electrical conductivity or impedance in at least one location, for example 2, 3, 4 or any larger number of locations within the blood vessels. In some embodiments, voltage values are acquired, for example as a way of estimating impedance. In some embodiments, changes in the values of at least one electrical parameter in each location over time and/or over a cardiac cycle are measured. Alternatively or additionally, the one or more electrodes of the pacing lead measure values of the tissue thickness or changes in tissue thickness in at least one location for example 2, 3, 4 or any larger number of locations within the blood vessels. In some embodiments, the tissue thickness values and/or the changes in tissue thickness values in each location are measured over time and/or over a cardiac cycle. In some embodiments, the position and/or the changes in position of the pacing lead, for example the position of pacing lead tip is determined based on the measured values. Optionally, the changes in position of the pacing lead are determined over time and/or over a cardiac cycle. For example, at least 2, at least 5, at least 10, at least 20 or smaller or intermediate numbers of samples are acquired per cardiac cycle.

According to some embodiments, the at least one electrical parameter values, thickness of the tissue and/or position of the pacing lead, for example pacing lead tip, are measured following electric field application, also termed herein as pacing. In some embodiments, the electric field is applied by at least one electrode positioned within the body, for example on the heart, near the heart or within blood vessels proximal to the heart. Alternatively, the electric field is applied by an electrode positioned outside the body, for example on the skin. In some embodiments, the electric field is applied epicardially, for example from within the coronary sinus (CS). Alternatively, the electric field is delivered endocardially. In some embodiments, the electrical field is applied to septal tissue of the heart, for example to deliver electric field to one or more branches of the bundles of His.

According to some embodiments, mechanical properties of the cardiac tissue are determined based on the values measured by the one or more electrodes of the pacing lead. In some embodiments, the mechanical properties of the cardiac tissue comprise the thickness or changes in thickness of the cardiac tissue over time or over a cardiac cycle. Optionally, the mechanical properties of the cardiac tissue comprise the thickness and/or changes in thickness following electric field delivery. Alternatively or additionally, the mechanical properties of the cardiac tissue comprise distances between different sites in the cardiac tissue, for example endocardial distances between endocardial sites, over a time period or over a cardiac cycle. In some embodiments, the mechanical properties of the cardiac tissue comprise distances between epicardiac sites, for example over a time period or over a cardiac cycle.

According to some embodiments, an anatomical map and/or a functional map of the cardiac tissue is generated based on the measured values or based on the changes in the measured values over time or over a cardiac cycle. Alternatively or additionally, an anatomical map and/or a functional map of the cardiac tissue is generated based on the changes in the pacing lead position over time and/or over a cardiac cycle. Optionally, the anatomical map and/or the functional map of the cardiac tissue is generated based on changes in the measured values following electric field application.

According to some embodiments, the functional map comprises electrical information and/or mechanical information on the mapped cardiac tissue, for example on an epicardial tissue, and/or on an endocardial tissue. Alternatively or additionally, the functional map comprises electrical and/or mechanical information on different heart regions, for example left ventricle electrical properties which comprise local activation map or local activation voltage of the left ventricle.

According to some embodiments, the generated anatomical map and/or the functional map are projected to a user, for example an operator, of the navigation and mapping system. In some embodiments, the system projects to the user based on the generated maps changes in the measured mechanical and/or anatomical properties over time and/or over different locations. Additionally and/or alternatively, the system projects to a user based on the generated maps changes in the measured mechanical and/or anatomical properties in response to delivery of an electric field, optionally delivered at selected locations, for example selected locations indicated on the generated map. In some embodiments, the system projects to the user, based on the generated maps changes in the measured mechanical and/or anatomical properties of the cardiac tissue between a pacing state, for example when an electric field is delivered to the tissue and a non-pacing state, and/or between different pacing modes.

According to some embodiments, the electric field is delivered to endocardial tissue or epicardial tissue. In some embodiments, the electric field is delivers to one or more branches of the bundle of His, for example by septal pacing.

According to some embodiments, a position for implantation of one or more pacing electrodes of a pacemaker device are selected based on the generated anatomical and/or functional maps of the cardiac tissue. Alternatively or additionally, the position for implantation of the one or more pacing electrodes is selected based on the information projected to the operator.

According to some embodiments, the pacing electrodes are positioned on epicardial tissue. In some embodiments, the pacing electrodes are positioned near or at the septum, for example to deliver an electric field to one or more branches of the bundle of His.

According to some embodiments, the position for implantation is optimized by delivery of an electric field at a selected implantation site and monitoring changes in the generated anatomical and/or functional maps following the electric field.

An aspect of some embodiments relates to determining a geometrical change of an anatomical region by measuring changes in position of two or more locations in elongated blood vessels, for example veins or arteries. In some embodiments, the two or more locations are within elongated blood vessels of the heart, for example the great cardiac vein and the great cardiac vein tributaries. In some embodiments, the anatomical region is positioned near or between the two or more locations. Alternatively or additionally, the two or more locations are within the anatomical region. In some embodiments, the anatomical region is positioned underneath or toward the cavity lumen, for example the left ventricle lumen, of at least one location.

According to some embodiments, changes in the position of the electrode lead at different locations within the blood vessel during a cardiac cycle are measured. Alternatively or additionally, changes in the local myocardial thickness in at least one location within the blood vessel, optionally during a cardiac cycle are measured. In some embodiments, the measured position and/or the measured changes in position, and/or the changes in the myocardial thickness are coordinated, also termed herein synchronized, with the cardiac cycle, for example to determine the position of the electrode lead in different phases of the cardiac cycle. In some embodiments, electrode lead locations exhibiting similar measured values are annotated as a single region. In some embodiments, a shape and size of the region is determined based on the measured changes in position during cardiac cycle.

According to some embodiments, changes in position are measured, for example by imaging. Optionally, the changes are measured in an inward direction, for example to determine a thickness of a tissue.

An aspect of some embodiments relates to imaging of arteries and/or veins by one or more electrodes of a pacing lead. In some embodiments, the pacing lead is advanced through the arteries and/or veins while recording values of at least one parameter of a tissue surrounding the pacing lead. In some embodiments, the arteries and/or veins comprise the coronary sinus and/or any other blood vessels connected to the coronary sinus. In some embodiments of the invention, the imaging is used to detect vascular abnormalities, such as aneurisms, stenosis, contradictions and geometry changes causes by extra-vascular elements and/or implants

According to some embodiments, the one or more electrodes of the pacing lead measure electrical parameter values of the tissue, for example impedance and/or electrical conductivity of the tissue surrounding the lead. Alternatively or additionally, the one or more electrodes of the pacing lead measure the thickness of the tissue, optionally based on the measured electrical parameter values. In some embodiments, the one or more electrodes measure the electrical parameter and/or the tissue thickness in at least one location within the blood vessels. Alternatively, the one or more electrodes measure the electrical parameter and/or the tissue thickness in at least two or more locations within the blood vessels, for example 2, 3,5,8 locations within the blood vessels.

Exemplary methods for estimating and/or measuring impedance based on measurements made at catheter electrodes are described in U.S. Provisional Patent Application No. 62/667,530 titled “MEASURING ELECTRICAL IMPEDANCE, CONTACT FORCE AND TISSUE PROPERTIES”.

According to some embodiments, the one or more electrodes measure changes in the electrical parameter values and/or the thickness of the tissue during a cardiac cycle. In some embodiments, the measured parameter values and/or thickness are synchronized with the cardiac cycle, for example to identify changes in the tissue electrical properties and/or thickness at different phases of the cardiac cycle.

In some embodiments of the invention, change in thickness is interpreted to mean muscle contraction (increase) and/or stretching of scar tissue (decrease).

According to some embodiments, a control unit connected to the pacing lead generates a functional and/or an anatomical map of the blood vessels and/or bifurcations of the blood vessels based on the measured electrical parameter values and/or the measured tissue thickness value, optionally during and/or in relation to a cardiac cycle, for example, by collecting data and positions and building a map. Optionally, the map is overlaid on a known or estimated vascular tree. In some embodiments, the functional map comprises electrical parameter(s) and/or mechanical parameter(s) of the blood vessels.

According to some embodiments, the generated functional and/or anatomical maps are presented to a user of a navigation system, for example on a display of the navigation system. In some embodiments, the generated functional and/or anatomical maps are presented to a user with information over a pre-determined time period and/or with functional and/or anatomical information measured at one or more locations within the blood vessels. In some embodiments of the invention, the display includes a model of at least part of the heart, so locations within the vasculature can be interpreted by a user as locations relative to the parts of the heart (e.g., left ventricle) or other organs.

An aspect of some embodiments relates to optimizing stimulation by using changes in tissue position information or tissue thickness for assessing expected and actual effect of stimulation. In some embodiments, position or changes in position or changes in thickness or a combination of the thickness and position at a specific location are measured following stimulation. Alternatively or additionally, the changes in position are detected at different locations following stimulation, for example to determine a time delay in the tissue response to the delivered stimulation. In some embodiments, the position is determined from within arteries and/or veins.

In some embodiments of the invention, stimulation is optimized by analyzing a map to identify locations where stimulation may be more beneficial.

According to some embodiments of the invention, there is provided tools to a physician to increase safety, efficiency and/or efficacy of cardiac therapies, for example cardiac resynchronization therapy.

According to some embodiments, a physician uses a pacing lead to be positioned in epicardial blood vessels, for example the epicardial vein, and connects at least two of its leads to a signal generator. In some embodiments, the signal generator causes transmission of distinctive signals from at least two electrodes of the pacing lead. In some embodiments, using these signals and optionally by knowing the distance between the two electrodes on the lead one can generate a map of the acquired voltages as a function of the distance between the two electrodes, for example using a dielectric imaging method. As used herein, dielectric imaging comprises using emissions and receiving electromagnetic signals in different frequencies. Dielectric imaging can be performed in multiple ways, common to all of them is the transmission and reception of electromagnetic signals from an imaging port. In some embodiments, the ensemble of locations and their distances from one another allows, for example to generate the localization of the location of the tip of the pacing lead.

According to some embodiments, a location of the electrodes are generated on an image, or on a series of images with known relative location. Alternatively, a positioning method is used, for example in relation to a reference which optionally is the series of images we acquired.

According to some embodiments, impedance navigation is used to locate the electrode of the pacing lead in a given electrical field. In some embodiments, the electric field is applied from within the patient body, for example using at least one electrode positioned within the body or optionally the lead itself. Optionally or alternatively, the electric field is applied by at least one electrode positioned outside the body, for example at least one electrode attached to the skin of the patient. Optionally, using these and other methodologies, an operator, for example a user of the navigation system navigates a pacing lead to a desired location based on a reconstructed image of the epicardial cardiac veins, generated by the navigation system.

According to one embodiment the sensed local electrical activation of the myocardium through the electrodes of the pacing lead or through a different mapping catheter are recorded and associated with their location on the vascular tree. Optionally, the application collects more than two activations to generate an epicardial local activation map.

According to some embodiments, a tissue surface, for example an epicardial tissue is extended between the veins and their branches. In some embodiments, the local activation times recorded from the veins are portrayed over the epicardial surface and are presented to the operator to help him identify areas of interest for delivering a treatment, for example CRT. In some embodiments, the operator is searching for the latest local epicardial electrical activation, for example for positioning the pacing lead. In some embodiments, for example in His-bundle pacing, the operator is seeking to implant the pacing lead close to the His bundle, which can be recorded on the endocardial side of the right ventricle.

According to another embodiment of the invention, as the lead is bounded by the vein diameter, the vein movements reflect the movement of the epicardium. In some embodiments, the system tracks the locations of the lead within the veins and optionally uses knowledge of the cardiac cycle (for example from an ECG signal that is acquired simultaneously), the momentary movement of the veins and their branches (which corresponds to the movement of the epicardium they are attached to) is captured with it respective phase of the cardiac cycle.

According to some embodiments, an algorithm identifies the lead location at more than one different phases of the cardiac cycle. In some embodiments, the algorithm calculates a distance between lead location of the same cardiac phase, for example to calculate the regional shortening/lengthening of the epicardium between the two or more locations of the epicardium bounded by at least two separate locations. In some embodiments, the algorithm calculates an area bounded by the adjacent veins, for example to derive a measure for the local contraction dilation of the epicardial area bounded by the veins.

According to some embodiments, epicardial local activation times are determined based on the electrograms measured by the pacing lead electrodes. In some embodiments, a corresponding time difference between the local epicardial activation time to the time of a location indifferent reference signal during the same heart cycle (for example the R wave of the ECG signal), allows for example to derive the epicardial local activation time. In some embodiments, an algorithm that analyze local mechanical deformation identifies more than one point during the cardiac cycle and optionally relates the identified points to the local activation time at that location.

According to some embodiments, regional mechanical alterations are processed, for example to identify a cardiac cycle phase at which the area bounded by the adjacent veins reaches a maximum value (e.g., local max dilation time). Additionally, regional mechanical alterations are processed, for example to identify a minimum value (local max shortening time). In some embodiments, the algorithm measures the time between the local electrical activation to the time of local max shortening time and optionally denotes this time difference as the electro mechanical coupling time (EMCT) between local activation and the ensuing local contraction. In some embodiments, during placement of left ventricular lead for treating patient with heart failure, the local activation time or the most delayed activation is determined, optionally by monitoring the impact of different pacing location of the ensuing EMCT.

Some embodiments of this invention have application beyond CRT lead implantation as well as for interventional treatment of coronary ischemic heart disease that will be described in different embodiments. In some embodiments of the invention, patients with structural heart disease or with coronary disease are treated. Some embodiments of the invention are used during the implantations of ICD, pacemakers and/or other stimulators.

Although some embodiments of the invention use an example of utilizing dielectric imaging for locating and mapping and tissue imaging in the coronary sinus tree, it should be noted that other methods for locating and/or imaging such as impedance and or magnetic navigation can be used instead.

Although one of the preferred embodiments is making use of the pacing lead as the tool to acquire the image and related mapping as well as perform the navigation with, in some embodiments one can use different tools than the pacing lead itself. In some embodiments, medical imaging is used to generate a volumetric image of the tissue composing a given volume. Optionally tissues can be differentiated one from the other, in one way they can be differentiated as normal or pathological tissues, in other examples, tissue can differentiated by their density, strength, composition, micro structure and other differentiators.

It is a purpose of some embodiments of the current invention to provide specific tools design, optionally optimized for dielectric imaging. More specifically, some embodiments of the current invention describe the design and operation of dielectric imaging tools both for medical and intrabody medical imaging.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Navigation and Mapping System

According to some exemplary embodiments, a control unit is electrically connected to one or more electrode leads, for example pacing electrode leads. In some embodiments, the control unit measures at least one electric parameter of a tissue, for example impedance, conductivity and/or electrical activity. In some embodiments, the control unit measures impedance, for example for tissue characterization. In some embodiments, the control unit measures electrical activity, for example to record heart contractions and cardiac cycle. based on signals received from the one or more electrode leads. In some embodiments, the at least one electric parameter is measured during the navigation of the pacing electrode leads within elongated blood vessels, for example arteries and or veins.

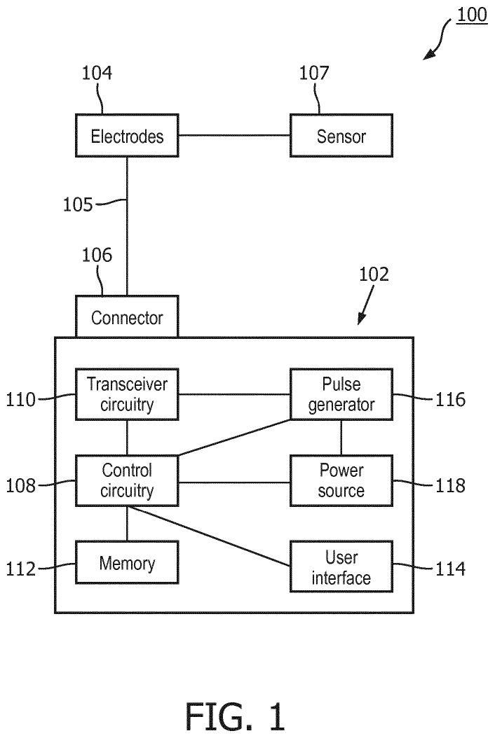

According to some exemplary embodiments, the at least one measured electric parameter is measured and used to determine one or more of position of the pacing electrode lead, location of blood vessel bifurcations and/or a preferred location for implantation of a pacing electrode, for example to deliver CRT. Alternatively, the position of the pacing electrode lead, location of blood vessel bifurcations and/or a preferred location for implantation of a pacing electrode are determined using information received from one or more position sensors on the lead, and/or using other navigation methods. Reference is now made to FIG. 1 depicting a navigation and/or mapping system, according to some exemplary embodiments of the invention.

According to some exemplary embodiments, a system for navigation and/or mapping, for example system 100 comprises a control unit, for example control unit 102 electrically connected to one or more electrodes, for example electrodes 104. In some embodiments, the electrodes 104 are electrically connected to the control unit 102 via an electrode lead, for example electrode lead 105. Optionally, two or more electrode leads, comprising at least one electrode on each of the electrode leads, are electrically connected to the control unit 102. In some embodiments, at least one sensor, for example position sensor 107 is connected to the electrodes 104. In some embodiments, each electrode lead comprises a position sensor, for example a position sensor 107, optionally at the tip of the electrode lead. In some embodiments, position information may be obtained from electrodes 104 solely or in addition to position information from position sensor 107.

According to some exemplary embodiments, the electrode lead 105 is electrically connected to the control unit 102 via at least one connector, for example connector 106. Optionally, the connector 106 is configured to allow electrical connection of two or more electrode leads to the control unit 102.

According to some exemplary embodiments, the control unit 102 comprises at least one control circuitry, for example control circuitry 108, electrically connected to the connector 106, via a transceiver circuitry, for example transceiver circuitry 110. In some embodiments, the control circuitry 108 receives one or more signals from the electrodes 104 through the transceiver circuitry 110. Additionally or alternatively, the control circuitry receives signals from the position sensor 107.

According to some exemplary embodiments, the control unit 102 comprises a memory 112, for example for storing the signals received from the electrodes 104 and/or from the position sensor 107. In some embodiments, the memory 112 stores one or more algorithms for determining the position of the electrode lead, the electrode, and/or the electrode tip based on signals received from the electrodes 104, and/or the sensor 107. Alternatively or additionally, the memory 112 stores information related to the cardiac cycle, for example information received from an ECG device.

According to some exemplary embodiments, the control unit 102 comprises a user interface 114 for delivery of indications and/or for receiving information from a user of the system. In some embodiments, the user interface 114 comprises at least one speaker and/or at least one display for example, for delivery of human detectable indications.

According to some exemplary embodiments, the control circuitry determines the position of the electrodes and/or identifies blood vessel bifurcations and/or maps heart regions, for example based on signals received from the electrodes and/or the position sensor 107 and using an algorithm stored in the memory 112. In some embodiments, the control circuitry 108 presents information to a user on a display of the user interface 114. Optionally, the information is presented on an anatomical, structural, and/or functional map stored in the memory 112. In some embodiments, the map is loaded into the memory 112 prior to the navigation of the electrode lead 105. Alternatively or additionally, the map is generated or updated by the control circuitry 108 during the navigation of the electrode lead 105.

According to some exemplary embodiments, the control circuitry 108 generates an anatomical and/or a functional of a cardiac tissue, for example an epicardial tissue or an endocardial tissue. In some embodiments, the anatomical and/or functional maps are generated based on signals received from electrodes 104 and/or sensor 107. In some embodiments, the maps are generated by the control circuitry 108 based on signals received from the electrodes 104 and/or sensor 107 over time and/or over a cardiac cycle. Alternatively or additionally, the maps are generated by the control circuitry based on signals received from the electrodes 104 and/or the sensor 107 from one or more locations within a blood vessel, for example an artery or a vein. In some embodiments, the maps are generated by the control circuitry 108 based on signals received from the electrodes 104 and/or sensor 107 following application of an electric field. In some embodiments, the generated functional maps comprise electrical and/or mechanical properties of the cardiac tissue. Optionally, the generated functional maps comprise changes in electrical and/or mechanical properties of the cardiac tissue over time, over cardiac cycle and/or following electric field application. In some embodiments, the maps generated by the control circuitry 108 and/or any imaging information based on the generated maps or the signals received from the electrodes and/or the sensor 107, is stored in memory 112.

According to some exemplary embodiments, the control unit 102 comprises at least one pulse generator, for example pulse generator 116, configured to generate an electric field, optionally, with parameters stored in the memory 112. In some embodiments, the electric field is delivered to a tissue, for example to a cardiac tissue by one or more of the electrodes 104. In some embodiments, the control circuitry 108 measures at least one parameter of the tissue following or during the delivery of the electric field. Optionally, the electric field is delivered by one or more electrodes positioned outside the blood vessels, for example on the skin of the patient. In some embodiments, the electric field is delivered, for example for imaging the tissue and/or for monitoring the contraction of different heart regions.

According to some exemplary embodiments, the control unit 102 comprises an electric power source, for example power source 118. In some embodiments, the power source 118 is configured to provide electrical power to the control unit 102, for example to the pulse generator 116 and/or to the control circuitry 108. Alternatively, the control unit 102 is electrically connected to an external power source.

### Exemplary Navigation and/or Mapping Process

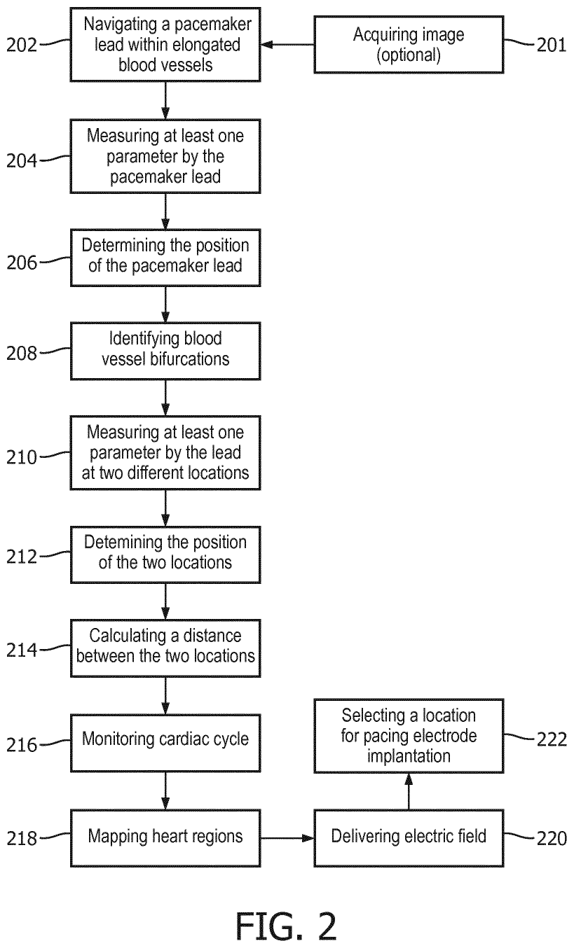

Reference is now made to FIG. 2 depicting a process for navigation and/or mapping of an anatomical tissue or organ, for example cardiac tissue, according to some exemplary embodiments of the invention. The process is described in a maximal manner, and it should be noted that some of the steps are optional and the order of the described steps can be changed.

According to some exemplary embodiments, an image is acquired at 201. In some embodiments, an image, for example an anatomical image and/or a functional image of an anatomical region, for example an anatomical region of the heart is acquired. In some embodiments, the image is acquired using an imaging technique, for example ultrasound imaging, magnetic resonance imaging (MRI), computed tomography (CT), x-ray and/or any angiography technique. Alternatively or additionally, a functional image of the anatomical region, for example a functional imaging generated by electrophysiological analysis, for example electrocardiogram (ECG) is acquired at 201. In some embodiments, a generated map is overlaid on the image.

According to some exemplary embodiments, one or more pacemaker electrode leads are navigated within the body at 202. In some embodiments, the electrode leads are navigated within blood vessels, for example arteries and/or veins. Optionally, the electrode leads are navigated to a position suitable for implantation of a pacing electrode for delivery of CRT. In some embodiments, the one or more electrode lead is navigated within the coronary sinus.

According to some exemplary embodiments, at least one parameter is measured by the one or more pacemaker leads at 204. In some embodiments, the at least one parameter comprises an electrical parameter, for example conductivity or impedance. In some embodiments, the electrical parameter is measured during the navigation of the one or more electrical leads. In some embodiments, the electrical parameter is measured at different locations within the blood vessels. Optionally, the electric parameter is measured by contacting the blood vessel wall.

Exemplary methods for estimating and/or measuring impedance are described in U.S. Provisional Patent Application No. 62/667,530 titled “MEASURING ELECTRICAL IMPEDANCE, CONTACT FORCE AND TISSUE PROPERTIES”.

According to some exemplary embodiments, a position of the pacemaker lead, for example a tip of the pacemaker lead optionally comprising one or more electrodes is determined at 206. In some embodiments, the position of the pacemaker lead is determined based on the values of the electrical parameter measured at 204. In some embodiments, the position of the electrode lead is determined based on signals received from at least one position sensor associated with the electrode lead, for example position sensor 107 shown in FIG. 1.

According to some exemplary embodiments, one or more blood vessels bifurcations are identified at 208. In some embodiments, the blood vessel bifurcations, for example bifurcations of the coronary sinus or blood vessels connected to the coronary sinus are identified at 208. In some embodiments, the blood vessel bifurcations are identified based on the measured values of the electrical parameter. In some embodiments, the bifurcations are identified by combining the measured values of the electrical parameter and additional information received from an imaging or an electrophysiological analysis.

According to some exemplary embodiments, the electrical parameter is measured by the electrode lead at two or more different locations at 210. In some embodiments, the electrical parameter is measured at two or more locations by navigating the electrode lead within the blood vessels to two or more different locations. Alternatively or additionally, the electric parameter is measured by two or more axially and/or radially spaced apart electrodes of the same electrode lead In some embodiments, at least some of the electrodes are positioned on a sheath of the pacemaker lead.

According to some exemplary embodiments, the position of the two locations is determined at 212. In some embodiments, the position of the two locations is determined based on the measured values of the electrical parameter. Alternatively, the position of the two locations is determined based on information received by one or more sensors on the electrode lead, for example a position sensor. Optionally, the position of the two locations is determined based on a combination between signals received from one or more electrodes of the lead and information stored in a memory, for example memory 112.

According to some exemplary embodiments, a distance between the two locations is calculated at 214. In some embodiments, the distance is calculated based on the determined position of the two locations. Optionally, the distance is calculated based on the determined locations and on an image and/or a map, for example an anatomical and/or a functional map of the anatomical region.

According to some exemplary embodiments, the cardiac cycle is monitored at 216. In some embodiments, the cardiac cycle is monitored by at least one electrode positioned within the body. Alternatively, the cardiac cycle is monitored by at least one electrode positioned outside the body, for example on the skin. In some embodiments, the cardiac cycle is monitored by one or more electrodes of an ECG device. In some embodiments, the cardiac cycle is monitored in a timed relationship to the measurement on the electrical parameter at 210, for example during the measurement of the electrical parameter.

According to some exemplary embodiments, the changes in position of each of the two locations during the cardiac cycle are determined. In some embodiments, the position of each location changes between a maximal value and a lower value. Optionally, the difference between the maximal and the minimal values is related to the ability of the tissue in the specific location to contract and/or to the ability of the tissue in the specific location to conduct electrical current.

According to some exemplary embodiments, one or more heart regions are mapped at 218. In some embodiments, the heart regions, for example epicardial regions are mapped at 218. In some embodiments, the heart regions are mapped based on the changes in distance between the two locations, that are optionally reside in the mapped heart regions, during a cardiac cycle. In some embodiments, the heart regions are mapped based on the difference between the maximal and minimal values. In some embodiments, mapping is performed by moving a pacemaker lead within blood vessels and measure the at least one parameter from within the blood vessel to determine position. Alternatively or additionally, mapping is performed by moving one or more electrodes within the pericardial sac.

According to some exemplary embodiments, heart regions are mapped according to their tissue type. In some embodiments, if the two locations demonstrate a small and/or a negative difference between the maximal and minimal values during a cardiac cycle, then a tissue region between the two locations includes a high percentage of scar tissue, and is optionally annotated as scar tissue. Alternatively, if the two locations demonstrate a high and/or a positive difference between the maximal and minimal values, then a tissue region between the two locations includes a high percentage of muscle tissue, and is optionally annotated as muscle tissue. In some embodiments, the size and/or the shape of the region between the two locations is determined, optionally based on grouping locations with similar difference values between the measured maximal and minimal values into a single region with a similar annotation.

According to some exemplary embodiments, an electric field is delivered to the cardiac tissue at 220. In some embodiments, the electric field is delivered as a mapping electric field, for example to allow mapping of the cardiac tissue. Alternatively or additionally, the electric field is delivered as a stimulating electric field, for example to evaluate the response of the cardiac tissue to stimulation at different locations. In some embodiments, the electric field is delivered to the cardiac tissue by one or more of the pacing lead electrodes. Alternatively, the electric field is delivered by different electrodes, for example one or more electrodes positioned on a different electrode lead. In some embodiments, the electric field is delivered in a timed relationship to the measuring of the at least one parameter at 204 or at 210, for example prior to the measuring. In some embodiments, the electric field is delivered with known parameter values, for example selected intensity and/or selected frequency. Optionally, the electric field parameter values are stored in the memory 112.

According to some exemplary embodiments, heart regions are mapped at 218, based on measurement following the electric field delivery, for example position measurements, distance between the two locations, contraction values of different anatomical regions following the electric field delivery. In some embodiments, a functional map is generated by combining the contraction timing or contraction delay at different locations into a single functional region. In some embodiments, the contraction timing or the contraction delay at a specific location following electric field delivery is calculated based on the changes in position of the electrode location following electric field delivery. In some embodiments, a tissue contraction delay map, for example an epicardial activation map is generated based on changes in position following electric field delivery.

According to some exemplary embodiments, a location for implantation of a pacing electrode is selected at 222. In some embodiments, the implantation location is selected based on the generated functional and/or anatomical maps, for example a generated tissue type map and/or the generated epicardial activation map.

### Exemplary Blood Vessel Bifurcations Identification

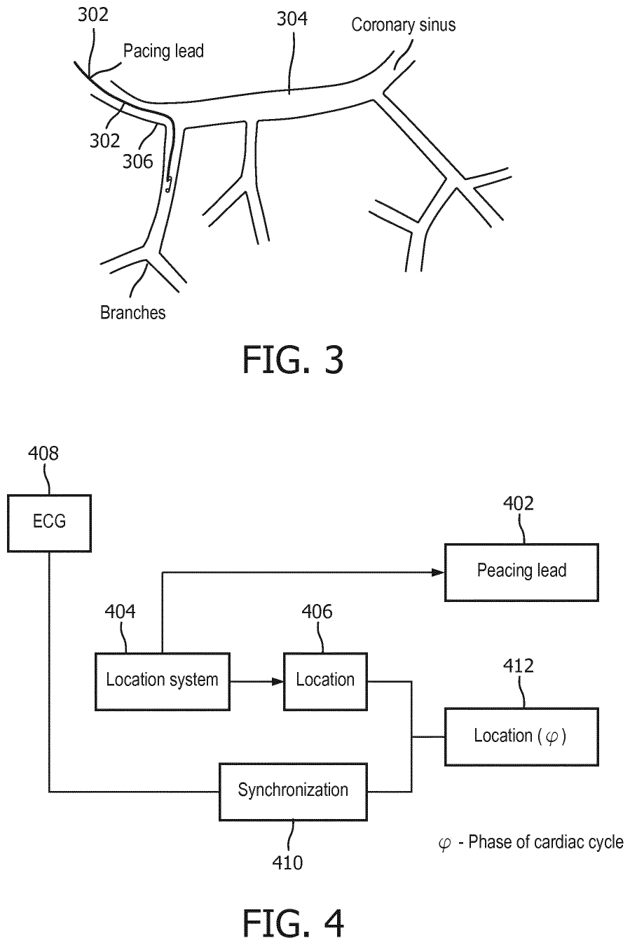

According to some exemplary embodiments, when navigating a pacing lead into a desired location, the lead is forwarded through blood vessels, for example arteries and/or veins, for example through the coronary sinus. In some embodiments, during the navigation, bifurcations in the blood vessels through which the pacing lead advances are identified, for example by determining the position of the pacing lead. Reference is now made to FIG. 3, depicting blood vessel bifurcations identification, according to some exemplary embodiments of the invention. In some embodiments, the bifurcations are identified using a generated image, and then the bifurcations position is used as a reference during the navigation process.

According to some exemplary embodiments, a pacing lead 302 is navigated through blood vessels, for example through the coronary sinus 304. In some embodiments, at least one electrical parameter is measured by one or more electrodes on the pacing lead, for example conductance and/or impedance of tissue. Alternatively, at least one sensor on the pacing lead 302, for example a position sensor measures the position of the pacing lead at different locations during navigation.

According to some exemplary embodiments, a control unit connected to the pacing lead, for example control unit 102 determines the position of the electrode lead. In some embodiments, based on the measured electrical parameter, changes in tissue type are identified, which allows, for example, to identify one or more bifurcation in the navigation path of the pacing lead, for example bifurcation 306. Optionally, the one or more bifurcations are identified by relating the signals received from the pacing lead or the determined position of the electrode to an anatomical map or a functional map, optionally stored in a memory of the control unit. In some embodiments, the stored maps are generated based on imaging analysis information.

### Exemplary Position Synchronization with Cardiac Cycle

According to some exemplary embodiments, position measurement in each location is synchronized with the cardiac cycle, for example to measure the time between the local electrical activation to the time of local max shortening time and to optionally denote this time difference as the electro mechanical coupling time (EMCT) between local activation and the ensuing local contraction. Reference is now made to FIG. 4, depicting a synchronization process between location measurement and the cardiac cycle, according to some exemplary embodiments of the invention.

According to some exemplary embodiments, one or more electrodes of a pacing lead 402 delivers signals to a location system 404. Alternatively at least one sensor, for example a position sensor on the pacing lead 402 delivers signals to the location system 404. In some embodiments, the location system 404, determines the location 406 of the pacing lead 402 or the location at least one electrode of the pacing lead.

According to some exemplary embodiments, a system for monitoring cardiac cycle, for example ECG 408, monitors cardiac cycle during the delivery of signals from the pacing lead. In some embodiments, the cardiac cycle information and the location information of the electrode are synchronized 410. In some embodiments, following synchronization, the location or position of the electrode at a selected cardiac cycle phase is determined at 412.

### Exemplary Regional Mapping

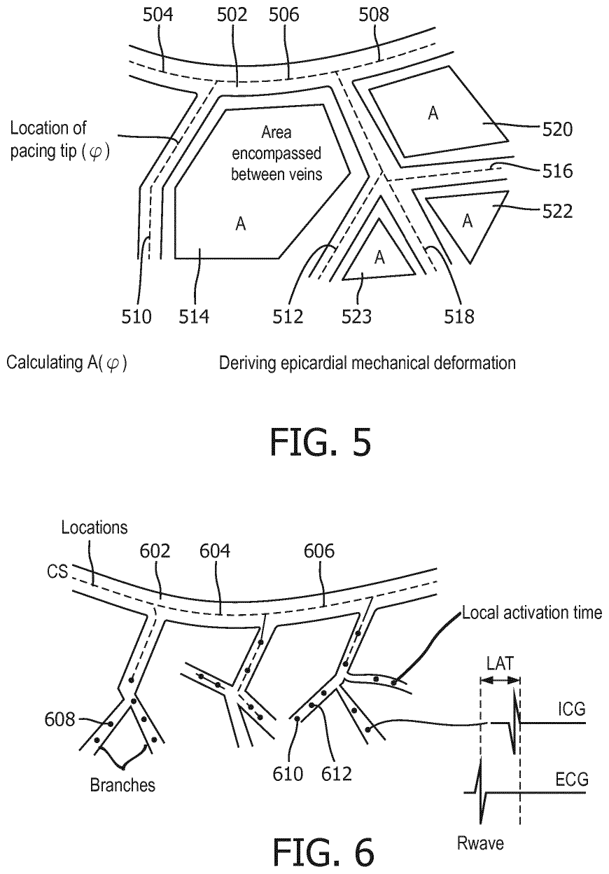

Reference is now made to FIG. 5, depicting mapping of regions in cardiac tissue, according to some exemplary embodiments of the invention.

According to some exemplary embodiments, a pacing lead is navigated through blood vessels, for example blood vessel 502. In some embodiments, the location of the pacing lead, for example the position of the pacing lead tip is determined at different locations within the blood vessel 502, as previously discussed. In some embodiments, by determining the position of the pacing lead and/or the changes in the position of the pacing lead, for example during cardiac cycle, a map of tissue types and/or a map of functional regions are generated.

According to some exemplary embodiments, position changes of the pacing lead are measured when the pacing lead is placed at a selected location within the blood vessel, for example locations 504, 508, 510, 506, 518, 516 and 512. In some embodiments, the changes in the position result from the movement of the cardiac tissue, for example epicardial tissue attached to the blood vessel. In some embodiments, the movement of the epicardial tissue is between a minimal contraction value and a maximal expansion value. In some embodiments, during mapping, pacing lead positions which exhibit similar differences between the minimal contraction value and the maximal contraction values are grouped into a single region, for example regions 514, 522, 520, 523.

According to some exemplary embodiments, each of the regions has a different tissue composition, disease state, incoming conduction or other contraction and/or excitation properties, which optionally affects the differences between the minimal contraction value and a maximal expansion value. In some embodiments, tissue regions which exhibit minor or negative differences include a high percentage of scar tissue. Alternatively, tissue regions which exhibit major difference between the minimal contraction value and a maximal expansion value include a high percentage of muscle tissue.

Reference is now made to FIG. 6, depicting measuring position of the pacing electrode at different locations within blood vessels following electric field delivery, according to some exemplary embodiments of the invention.

According to some exemplary embodiments, the pacing lead is navigated within a blood vessel 602, and the position of the pacing lead is determined at different locations, for example locations 604 and 606. Additionally, the changes in position of the pacing lead in each location is determined, following delivery of an electric field to the tissue, for example at locations 608, 610 and 612. In some embodiments, the response of the tissue to the delivered electric field is calculated, based on the determined changes in position. In some embodiments, and without being bound by any theory, different cardiac tissues respond in a different time delay to a delivered electric field, for example based on their distance from the electric field delivery site or other electrical properties of the tissue.

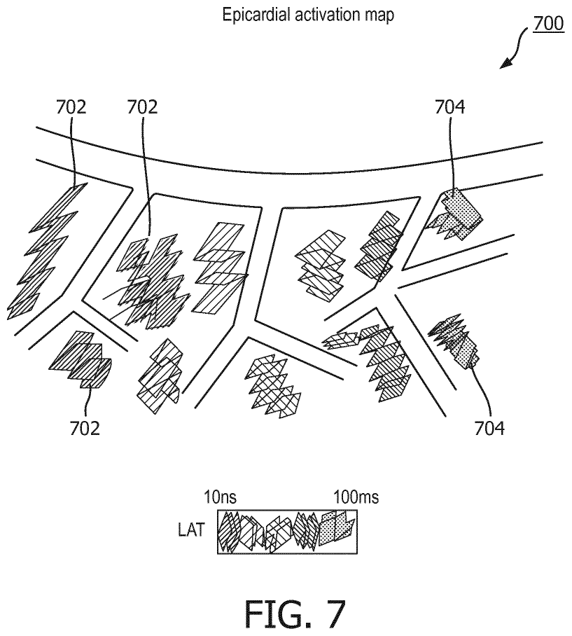

Reference is now made to FIG. 7, depicting an epicardial activation map, according to some exemplary embodiments of the invention.

According to some exemplary embodiments, based on the measured delay between different tissue regions, for example as described in FIG. 6, a functional map is generated, for example activation map 700. In some embodiments, tissue regions which exhibit similar contraction time delay are grouped together or have the same annotation, for example in activation map 700 tissue regions that exhibit a contraction time delay of 10 ns for example regions 702. In some embodiments, tissues that exhibit a time delay of 100 ms for example regions 704.

According to some exemplary embodiments, an implantation position of a pacing electrode is selected, based on the activation map shown in FIG. 7, for example to allow efficient contraction synchronization between the different tissue regions. In some embodiments, an implantation position having a desired delay relative to other electrode is selected. In some embodiments, using the activation map shown in FIG. 7, multiple pacemaker electrodes are positioned. Optionally, the measured activation time or other cardiac cycle local properties are used to select starting parameter values for pacing.

It is expected that during the life of a patent maturing from this application many relevant pacing leads will be developed; the scope of the term pacing lead is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Some examples of some embodiments of the invention are listed below:

Example 1. A method for navigating a pacing lead within blood vessels, comprising:

advancing a pacing lead within elongated blood vessels;

measuring values of at least one parameter of a tissue surrounding said pacing lead at one or more locations within said blood vessels;

determining a position of said pacing lead within said blood vessels based on said measured values.

Example 2. A method according to example 1, comprising:

identifying one or more bifurcations in said blood vessels based on said measured values.

Example 3. A method according to any one of the previous examples, wherein said elongated blood vessels comprise coronary arteries and/or veins.
Example 4. A method according to any one of the previous examples, comprising:

synchronizing said measured parameter values with cardiac activity.

Example 5. A method according to example 4, wherein said determining comprises determining a position of said pacing lead in relation to said cardiac activity.
Example 6. A method according to any one of the previous examples, wherein said at least one parameter of a tissue comprises tissue conductance and/or tissue impedance.
Example 7. A method for mapping an anatomical region, comprising:

advancing a pacing lead within a bodily cavity;

measuring values of at least one parameter of a tissue surrounding said pacing lead at one or more locations within said bodily cavity;

mapping said anatomical region based on said measured values.

Example 8. A method according to example 7, wherein said measuring comprises measuring values of said at least one parameter over time.
Example 9. A method according to any one of examples 7 or 8, comprising:

monitoring a cardiac cycle;

calculating changes in said measured values in relation to the cardiac cycle;

wherein said mapping comprises mapping said anatomical region according to said calculated changes.
Example 10. A method according to any one of examples 7 or 8, comprising:

delivering an electric field to a cardiac tissue;

calculating changes in said measured values before and after said delivering;

wherein said mapping comprises mapping said anatomical region according to said calculated changes.

Example 11. A method according to any one of examples 7 to 10, wherein said mapping comprises generating an anatomical and/or a functional map of said anatomical region.
Example 12. A method according to example 11, wherein said functional map comprises mechanical and/or electrical map of said anatomical region.
Example 13. A method according to any one of examples 7 to 12, wherein said anatomical region comprises epicardium, endocardium, and/or left ventricle of the heart.
Example 14. A method according to any one of examples 7 to 13, wherein said bodily cavity comprises arteries and/or veins and/or pericardial sac.
Example 15. A method according to any one of examples 7 to 14, wherein said at least one parameter comprises impedance, electrical conductivity and/or thickness.
Example 16. A method for determining a heart function, comprising:

positioning a pacing lead in at least one location in contact with a heart wall;

measuring values of at least one parameter in said at least one location; determining a heart function at said at least one location based on said measured parameter values.

Example 17. A method according to example 16, wherein said measuring comprises measuring values of said at least one parameter in said at least one location during cardiac cycle.
Example 18. A method according to any one of examples 16 or 17, wherein said positioning comprises positioning said pacing lead in two or more locations contacting said heart wall, and wherein said measuring comprises measuring said at least one parameter in said two or more locations.
Example 19. A method according to any one of examples 16 to 18, comprising delivering an electric field to a cardiac tissue; wherein said measuring comprises measuring said values of said at least one parameter before and after said delivering.
Example 20. A method according to example 19, wherein said delivering comprises delivering said electric field to said cardiac tissue with at least one set of pacing parameters, comprising delay from one or more additional left ventricular leads, voltage, latency.
Example 21. A method according to any one of examples 16 to 20, wherein said heart function comprises mechanical and/or electrical heart function.
Example 22. A method according to any one of examples 16 to 21, comprising generating a functional map of one or more anatomical regions of the heart based on said determined heart function.
Example 23. A method for determining a geometrical change of an anatomical region, comprising:

positioning a pacing electrode in at least two different locations within an elongated blood vessel;

measuring values of at least one parameter of a tissue surrounding said pacing electrode in each of said at least two different locations;

determining a relative position of said two different locations based on said measured values;

calculating a change in said determined position over a period of time in said two different locations;

determining a geometrical change of an anatomical region between said two different locations based on said calculated change in said determined position.

Example 24. A method for selecting an implantation position of a pacing electrode, comprising:

positioning a pacing lead in at least one location near or within cardiac tissue;

delivering an electric field to a cardiac tissue;

measuring values of at least one parameter of a cardiac tissue surrounding said pacing lead in said at least one location before and after said delivering;

determining changes in said measured values before and after said delivering;

selecting an implantation position for a pacing electrode based on said determined changes.

1. A method for selecting an implantation position of a pacing electrode of a pacing lead within an anatomical region of a body, comprising:
measuring values of at least one parameter of an anatomical region surrounding the pacing lead in at least one location;
delivering an electric field to a tissue of the anatomical region;
measuring values of at least one parameter of the tissue surrounding the pacing lead in the at least one location after said delivering;
determining changes in said measured values before and after said delivering;
selecting an implantation position for the pacing electrode based on said determined changes.

2. A method according to claim 1, wherein the anatomical region comprises a region of the cardiovascular system, preferably the epicardium, endocardium, coronary vein and/or left ventricle of the heart.

3. A method according to claim 1, wherein the at least one parameter comprises impedance, conductivity and/or thickness.

4. A method according to claim 1, further comprising generating an anatomical and/or functional map of the anatomical region and selecting the implantation position of the pacing electrode based on said anatomical and/or functional map.

5. A method according to claim 4, wherein the generating of the anatomical and/or functional map comprises:
moving the pacing lead within a bodily cavity of the anatomical region;
measuring values of at least one parameter of a tissue surrounding said pacing lead at one or more locations within said bodily cavity over time;
calculating changes in said measured values before and after said delivering
mapping said anatomical region based on said calculated change of said measure value.

6. A method according to claim 1, further comprising determining a geometrical change of the anatomical region and selecting the implantation position of the pacing electrode based of said geometrical change.

7. A method according to claim 6, wherein the determining of the geometrical change comprises:
measuring values of the at least one parameter of a tissue surrounding the pacing electrode in at least two different locations within the anatomical region;
determining a relative position of said two different locations based on said measured values;
calculating a change in said determined position over a period of time in said two different locations;
determining a geometrical change of the anatomical region between said two different locations based on said calculated change in said determined position

8. A method according to claim 1, the method further comprising synchronizing said measured parameter values with cardiac activity of the body and selecting the implantation position of the pacing electrode based on said synchronized parameter.

9. A method according to claim 8, wherein said determining comprises determining a position of said pacing electrode in relation to said cardiac activity.

10. A method according to claim 1, wherein the measuring is done by one or more electrodes coupled to the pacing lead.

11. An apparatus for selecting an implantation position of a pacing electrode of a pacing lead within an anatomical region, the apparatus comprising a processor circuit in communication with the pacing lead, wherein the processor circuit is configured to:
measure values of at least one parameter of a tissue surrounding the pacing lead in at least one location within the anatomical region;
deliver an electric field to a cardiac tissue;
measure values of at least one parameter of the tissue surrounding the pacing lead in the at least one location within the anatomical region after said delivering;
determine changes in said measured values before and after said delivering;
select an implantation position for the pacing electrode of the pacing lead based on said determined changes.

12. An apparatus according to claim 11, wherein the processing circuit is configured to generate an anatomical and/or functional map of the anatomical region and selecting the implantation position of the pacing electrode based on said anatomical and/or functional map.

13. An apparatus according to claims 11, wherein the processing circuit is configured to determine a geometrical change of the anatomical region and selecting the implantation position of the pacing electrode based of said geometrical change.

14. A system for selecting an implantation position of a pacing electrode within an anatomical region of a body, wherein the system comprises:
the apparatus according to claim 11; and
the pacing lead coupled to at least a pacing electrode.

15. A computer program product comprising a computer readable medium having computer readable code embodied therein, the computer readable code being configured such that, on execution by a suitable computer or processor, the computer or processor is caused to perform the method of claim 1.
